(19) United States
(12) Patent Application Publication
(10) Pub. No.: US 2008/0200648 A1
(43) Pub. Date: Aug. 21, 2008

(54) Method of Peptide Synthesis

(75) Inventors: Matthieu Giraud, Sion (CH); Michaela Williner, Glis (CH); Oleg Werbitzky, Veyras (CH)

Correspondence Address: NIXON & VANDERHYE, PC, 901 NORTH GLEBE ROAD, 11TH FLOOR, ARLINGTON, VA, 22203, US

(73) Assignee: LONZA AG, Basel (CH)

(21) Appl. No.: 11/632,448

(22) PCT Filed: Jul. 13, 2005
(86) PCT No.: PCT/EP2005/007625

§ 371 Date: Jul. 23, 2007

(30) Foreign Application Priority Data
Jul. 16, 2004 (EP) 04016776.9
Jul. 20, 2004 (EP) 04017036.7
Jul. 20, 2004 (EP) 04017037.5

(51) Int. Cl. C07K 1/107

(52) U.S. Cl. 530/345

(57) ABSTRACT

A novel for amidation of C-terminal carboxyl groups of peptides is devised, which methods avoids undesired epimerisation of the α-carbon of the C-terminal amino acid yielding diastereoisomeric variants of the amidated peptide.

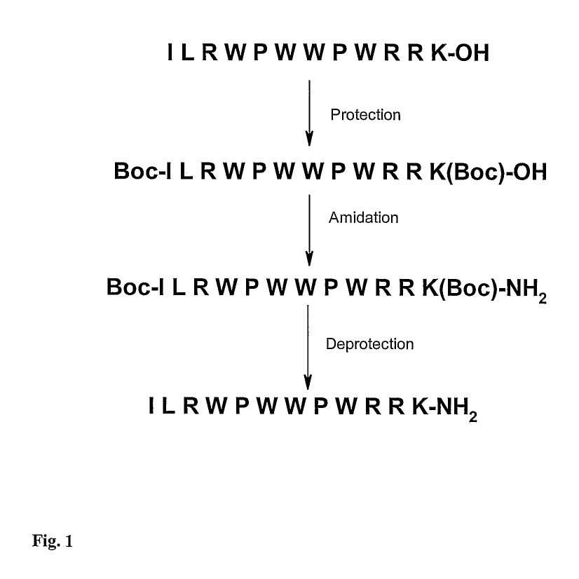

The present invention relates to the field of pharmaceutically useful polypeptides, namely to a respective method of chemical derivatization enhancing biological activity of such peptides. It devises a method of chemically amidating the C-terminus of a polypeptide.

Small peptide drugs have gained importance as a distinct, useful class of pharmaceutically active ingredients. For instance, anti-infective peptides, particularly cationic peptides whose industrial biotechnological manufacture is devised in US2003/0219854 A1, are a new class of broad spectrum antimicrobial substances which may help to combat the rapid spread of multi-drug resistance towards standard antibiotics amongst pathogenic microbes.

Naturally occurring anti-infective peptides include post-translational modifications such as C-terminal amidation that are paramount for sustained biological activity (Boman, Immunol. Rev. 173:5, 2000). For instance, C-terminal amidation eliminates potential charge and further protects from rapid degradation by ubiquitous exopeptidases. Whereas fully chemical synthesis may suitably introduce C-terminal amidfunctions during synthesis (e.g. Han et al., J. Org. Chem. 1996, 61,6326-6339; Albericio, F. and Barany, G., Int. J. Peptide Protein Res. 30, 1987, 206-216), the more cost-effective biotechnological route processing single peptide from large head-to-tail peptide concatemers lacks this possibility. Hence a biotechnologically manufactured peptide according to US 2003/0219854 A1 must be C-terminally amidated in a downstream processing step. Chemical amidation approaches described so far entailed a considerable degree of racemisation of the peptide backbone though.

US 2003/0219854 describes terminal amidation of a tryptophan-rich, Boc-protected 12-mer by reaction with an excess of ammonia and a base reagent (20 eq) in aprotic solvent, DMF, in the presence of near stoichiometric amounts (6 eq.) of an uronium coupling reagent (HATU: O-(1H-9-azabenzotriazole-1-yl)-1,1,3,3-tetramethyluronium hexafluorophosphate) and of the co-activator N-hydroxy-9-azabenzotriazole. The alleged yield of the amidated, deprotected peptide derivative was 47%.

Careful reproduction of this reaction sequence and reaction product analysis (s. exp. section) shows that the low yield is mainly due to unwanted epimerisation of the C-terminal amino acid, giving rise to two amidated diastereomers in almost 1:1 ratio as confirmed by electropherograms obtained with both a Tween 20 and a Cyclodextrin CE methods. This ratio obtained was also confirmed by a reverse-phase LC/ESI-MS tandem chromatography using Marfey's reagent as a chiral derivatizing agent of the hydrolyzed peptide as described in detail in the experimental section of the present application. That is half of the product is lost upon amidation due to efficient racemisation of the C-terminal amino acid.

Another method applicable only in the context of solid-phase synthesis is described in Albericio, F. and Barany, G. , Int. J. Peptide Protein Res. 30, 1987, 206-216. The method is however intricate in requiring preparation of special FMOC-trisalkoxy-benzylamide handles for functionalisation of the resin support. After coupling and synthesis of the peptide to the support, cleavage from the thus modified resin support yields readily amidated peptide at about 80% optical purity of the desired diastereisomer or diastereomer, which adds to a lower cleavage efficienty to give a total yield only of about 60 to 70% of the desired amidated diastereoisomer of the peptide. A similar approach with chemically slightly different amid-handles is described by the same author in U.S. Pat. No. 5,306,562.

The object of the present invention is to avoid the disadvantages of the prior art and to devise another or improved method of chemical, non-enzymatic amidation of a non-protected α-carboxyl group of an peptide or amino acid. According to the present invention, this object is solved by a method for amidating the free α-carboxyl group of an amino acid or peptide, comprising a first step of reacting said amino acid or preferably said peptide with a peptide coupling reagent in an organic solvent in the presence of a base and further in the presence of an anuonium salt of at least one peptide coupling additive wherein the ammonium cation is selected from the group consisting of ammonia, primary amine and secondary amine and wherein the side chain and α-amino function of said amino acid or peptide are protected with non-base-labile protection groups.

It is to be understood that the base reagent and the ammonium cation of the salt preferably are not identical and hence are not forming a conjugated acid-base pair. Rather, that there is a first base and a second base which second base is the ammonium cation according to the present invention. In the following, the term ‘base’ shall always be construed as to refer to said first base only. The second base will always be referred to as the ammonium compound or cation.

The method of the present invention has the advantage of minimizing adverse epimerisation of the α-carbon atom of the amino acid residue that is actually amidated at its α-carboxyl group. In di- or higher n-mer peptide, this amino acid residue is the C-terminal residue of a peptide which is a particularly preferred embodiment of the present invention.

The peptide or polypeptide according to the present invention may be any peptide. The protection of side chains and α-amino function will allow of achieving a sufficient solubility of a suitably protected peptide or amino acid in the organic solvent. It goes without saying that the solvent conditions according to the present invention prove favorably for efficient amidation under retention of configuartion, but may prove denaturing in view of leaving secondary or even tertiary structure of longer peptides unharmed, especially were such secondary or tertiary structure is not stabilized by covalent intrachain bonds or similar, possibly non-natural structural elements. Preferably, a peptide according to the present invention comprises 100 or less amino acid residues, more preferably 50 or less amino acid residues, most preferably 20 or less amino acid residues. This definiton includes peptides comprising non-natural composites such as D-amino acids, L- or D- amino acids with modified or unusual side chains or eventual non-amino-acid building blocks linking different parts of the peptide chain within afore said peptide. Solid-phase synthesis allows of efficient synthesis of peptides close to 50 residues, though further liquid phase segment condensation reaction allow of producing even more lengthy fully synthetic peptides. Now length restriction exists for peptides obtained from biotechnological production. Reduction of the water contents of the reaction mixture, as well as suitably excluding other protic solvents, in particular excluding beside water lower C1-C5 alkyl alcohols such as methanol, isopropanol or ethanol, is preferred for the present invention.

Preferably, the peptide is an antimicrobial or anti-infectious, bactericidal peptide, preferably is a cationic antimicrobial peptide such as e.g. ILRWPWWPWRRK, indolicidin, which are more active in their C-terminally amidated form. Anti-infective peptides, particularly cationic peptides such as indolicidin derivatives and in particular ILRWPWWPWRRK whose industrial biotechnological manufacture is devised in US2003/0219854 A1, are a new class of broad spectrum antimicrobial substances which may help to combat the rapid spread of multi-drug resistance towards standard antibiotics amongst pathogenic microbes.

Preferably the amidation reaction is carried out having a water contents of less than 25% (v/v), more preferably less than 15% (v/v), most preferably less than 5% (v/v). In a further preferred embodiment, the reaction is carried out under essentially water-free conditions. This may include using e.g. freshly distilled solvent or using protective nitrogen atmosphere.

Preferably the organic solvent is an aprotic organic solvent, more preferably it is a polar aprotic organic solvent. Suitable examples are acetonitrile, dimethylsulfoxid, dichloro-methane and N,N-dimethyl-formamide. Most preferably the organic aprotic solvent is N,N-dimethylformamide.

Preferably the amidation reaction is carried out of from −15° C. to 50° C. and that the pH is controlled during reaction to be in the range of about pH 8 to 9, preferably is controlled at about pH 8.5.

The type of reagents referred to above such as coupling agents, coupling additives and protection groups are well-known from standard peptide synthesis, which in essence is an amidation reaction, and are described in detail e.g. in Bodansky, M. , Principles of Peptide Synthesis, 2^{nd} ed. Springer Verlag Berlin/Heidelberg, 1993).

Non-base-labile protection groups are well-known in the art, the term base-labile being construed as to refer to abstraction at basic pH and/or aminolysis by primary or secondary amines as is common in the art. Suitable examples are e.g. 2-nitro-methoxyphenylsulfenyl-, Aloc(allyloxycarbonyl)-, Z(benzoxycarbonyl)-, Boc(tert.-butyloxy-carbonyl)-, Bpoc-(biphenyl-yl-isopropyloxy-carbonyl)-group. Orthogonal protection schemes including base labile protection are of course excluded from the present invention. Whether global protection with a single type of protection group or protection by different types of protection groups in the same peptide or amino acid is required, depends the source of the peptide from chemical or biotechnological synthesis and the type of side chains comprised in the peptide or amino acid. Notably, removal of such non-base labile groups may be effected by different means or under different reaction conditions. For instance, the Nps (o-nitro-phenylsulfenyl-) group is favorably removed by sulfihydryl nucleophils, Z-groups are removed by hydrogenolysis or Alloc groups may be removed by Pd(I)-catalysed hydrogenation. According to the present invention, suitably non-base labile protection groups are preferably removed after amidation by acidolysis as e.g. feasible for Boc-, Z-, trityl-, Nps- or Bpoc-groups.

From the gist of the present invention, it is apparent that protection of aspartyl- and glutamyl-side chains that might be present in the peptide according to the present invention deserves special attention and requires suitable carboxyl protection groups for selective protection or masking of ω-carboxyl groups leaving the C-terminal α-carboxyl group. This can be achieved, for instance, by global esterification of carboxyl-groups with a benzyl-halogenide to yield a benzylester followed by regioselective cleavage of the α-ester with LiOH in acetone (Bryant, P. et al., 1959, J. Chem. Soc., p. 3868 ff.) or by selective esterification of ω-carboxyl groups with alkyl-halogenides in the presence of Cu(II) salts masking the α-function during esterification by complex formation (Ledger, R., 1965, Austral. J. Chem. 18:1477ff); deprotection of the ω-carboxylgroup may also be facilitated by Cu(II) catalysis (Prestidge, R., 1975, J. Org. Chem. 40:3287ff.).

In another preferred embodiment of the present invention, the peptide or amino acid to be amidated is free from carboxyl groups other than the C-terminal α-carboxyl group to be amidated, preferably is free from glutamyl- or aspartyl residues.

Given that eventually the generation of glutaminyl- or asparaginyl-residues by concomittant ω-carboxyl groups is desireable and has been taken into account in the overall synthetic strategy of a peptide or amino acid according to the present invention, it is a further independent object of said invention to devise a method for amidating the free α-carboxyl group of an amino acid or peptide, comprising the step of reacting said amino acid or peptide with a peptide coupling reagent in the presence of a first base and further in the presence of an ammonium salt of of at least one peptide coupling additive wherein the ammonium cation is selected from the group consisting of ammonia, primary amine and secondary amine and wherein the side chain and α-amino function of said amino acid or peptide are protected with non-base-labile protection groups with the exception of the ω-carboxyl groups of individual aspartyl- or glutamyl-residues and wherein said α-carboxyl groups are amidated concomittantly with the free α-carboxyl group in the reaction. This second object is particularly preferred when using ammonia for the salt compound for obvious reason of yielding natural amino acid amides, namely asparaginyl and/or glutaminyl.

All technical explanations and descriptions of preferred embodiments of the invention given above or in the following sections and which relate to the first object of the invention extend likewise to this second object unless apparently incompatible with such further second object of the present invention, e.g. when claiming the complete absence of any aspartyl- or glutamyl-residue in the peptide.

Coupling reagents for peptide synthesis are well-known in the art (see e.g. Bodanszky, supra). Coupling reagents may be mixed anhydrides (e.g. T3P: propane phosphonic acid anhydride) or other acylating agents such as activated esters or acid halogenides (e.g. ICBF, isobutyl-chloroformiate), or they may be carbodiimides, activated benzotriazin-derivatives (DEPBT: 3-(diethoxyphosphoryloxy)-1,2,3-benzotriazin-4(3H)-one) or uronium or phosphonium salt derivatives of benzotriazol. In one preferred embodiment of the present invention, the coupling reagent is a coupling reagent other than a carbodiimide.

In a preferred embodiment, the coupling reagent is selected from the group consisting of uronium salts, phosphonium salts which have been found to give best total yields and best protection against racemization in the method of the present invention.

Further, more preferred is that the coupling reagent is selected from the group consisting of uronium salts and phosphonium salts of the benzotriazol capable of activating said α-carboxyl group and that the reaction is carried out in the presence of a base. Suitable and likewise preferred examples of such uronium or phosphonium coupling salts are e.g. HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazol-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), HAPyU (O-(benzotriazol-1-yl)oxybis-(pyrrolidino)-uronium hexafluorophosphate.

Preferably, the base or first base is a weak base whose conjugated acid has a pKa value of from pKa 7.5 to 15, more preferably of from pKa 7.5 to 10, with the exclusion of an α-amino function of a peptide or amino acid or amino acid derivative, and which base preferably is a tertiary, sterically hindered amine. Examples of such and further preferred are Hünig-base (N,N-diisopropylethylamine), N,N′-dialkylaniline, 2,4,6-trialkylpyridine or N-alkyl-morpholine with the alkyl being straight or branched C1-C4 alkyl, more preferably it is N-methylmorpholine or collidine (2,4,6-trimethylpyridine), most preferably it is collidine. All preferred embodiments described above and below are particulary preferred being worked in combination with a first weak base reagent as described in this section.

In another preferred embodiment, the amidation method is carried out with a carbo-diimide as the coupling reagent and preferably and particularly is carried out in the presence of a tertiary amine. More preferably, the carbo-diimide coupling reagent is selected from the group consisting of diisopropyl-carbodiimide, dicyclohexyl-carbodiimide and 1-ethyl-3-(3-dimethylaminopropyl)-carbodiimide), most preferably is 1-ethyl-3-(3-dimethylaminopropyl)-carbodiimide. Carbodiimides are for technical reasons less suited for industrial upscaling of synthetic processes, though. Preferably, the reaction using a carbodiimide as the coupling reagent is carried out in the presence of a second coupling additive other than said ammonium salt which second additive is a protonated, i.e. non-ionic N-hydroxy benzotriazol or N-hydroxy benzotriazol derivative compliant with the immediately below given definitions.

The use of coupling additives, in particular of coupling additives of the benzotriazol type, is also known. Hence it is further preferred that the coupling reagent additive is a nucleophilic hydroxy compound capable of forming activated esters, more preferably having an acidic, nucleophilic N-hydroxy function wherein N is imide or is N-acyl or N-aryl substituted triazeno, most preferably the coupling additive is a N-hydroxy-benzotriazol derivative (or 1-hydroxy-benzotriazol derivative) or is an N-hydroxy-benzotriazine derivative. Such coupling additive N-hdroxy compounds have been described in large and wide in WO 94/07910 and EP-410 182 and whose respective disclosure is incorporated by reference hereto. Examples are e.g. N-hydroxy-succinimide, N-hydroxy-3,4-dihydro-4-oxo-1,2,3-benzotriazine, 1-hydroxy-7-azabenzotriazole and N-hydroxy-benzotriazole. N-hydroxy-benzotriazine derivatives are particularly preferred, in a most preferred embodiment, the the coupling reagent additive is hydroxy-3,4-dihydro-4-oxo-1,2,3-benzotriazine.

Ammonium salt compounds of coupling additives are known and have been described, for instance in U.S. Pat. No. 4,806,641.

In a further particularly preferred embodiment, the uronium or phosphonium salt coupling reagent is an uronium salt reagent and preferably is HCTU, TCTU or HBTU and even more preferably is used in the reaction in combination with an ammonium salt of N-hydroxy-3,4-dihydro-4-oxo-1,2,3-benzotriazine.

In the context of the present invention, it is to be noted that HCTU and TCTU are defined as to be encompassed by the term ‘uronium salt reagent’ despite that these compounds and its analogues of formula I have been shown by means of crystal structure analysis to comprise an isonitroso moiety rather than an uronium moiety (O. Marder, Y. Shvo, and F. Albericio “HCTU and TCTU: New Coupling Reagents: Development and Industrial Applications”, Poster, Presentation Gordon Conference February 2002), an N-amidino substituent on the heterocyclic core giving rise to a guanidium structure instead. Hence such class of compounds according to formula I is termed guanidium-type subclass of uronium salt reagents according to the present invention:

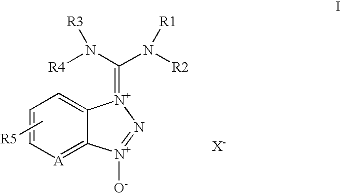

wherein R1, R2, R3, R4 each are alkyl, preferably are independently ethyl or methyl and wherein atom A is N or C and wherein R5 is H or, preferably, is an electron-withdrawing substitutent, more preferably is chloro, and wherein preferably X is a complex anion, more preferably is hexafluorphosphate or tetrafluoroborate.

As for the ammonium cation to be used in the salts of the present invention, the ammonium cation is ⁺H₂NR1R2 with R1,R2 each independently being H or being C1-C10, preferably being C1-C5, aliphatic or, alone or together, alicyclic hydrocarbon that may optionally further be substituted with aryl, alkoxy, aralkoxy, alkylaryl, aryloxy, hydroxy or halogen, preferably excluding N-heteroaromatic moieties. Preferably, R1, R2 is not further substituted and is independently alkyl as defined above. More preferably, the R1 is H and R2 is methyl, ethyl, propyl or isopropyl or R1,R2 are both H, ie the cation is NH₄⁺. NH₄⁺ is most preferred.

Preferably in a second step after the first reaction step, the amidated amino acid or peptide is isolated and in a third step is deprotected to yield the free side chains and α-amino function, preferably the amidated amino acid or peptide is partly or entirely deprotected by acidolysis. In one possible though less preferred embodiment of the present invention, an amino acid is amidated in this way and the pure L-amino acid carboxamid is obtained, suitably retaining side chain masking or protection groups, for use of such amino acid amid in excess in a normal segment condensation peptide synthesis scheme with a second peptide fragment. In this way, the more expensive peptide as arising e.g. from solid phase synthesis is spared from unnecessary losses due to amidation caused racemisation whilst the more cheaply available amino acid amid may be used in large excess for efficient coupling. Such embodiment is particularly suited for use of ammonia or lower alkyl amines as described above in the amidation reaction according to the method of the present invention.

In a further preferred embodiment, the protected peptide of the present invention is coupled to a conventional resin support by way of side chain anchoring to the functional linker moiety of the resin. Hence the C-terminus is left free for the purpose of amidation. Such support may be any resin commonly used in the art such CTC, Wang or Merrifield resin. In this way, the method of the present invention allows of efficient recovery and deprotection of the amidation product.

Further preferred is that in a second step after the first reaction step, the amidated, protected amino acid or preferably peptide is isolated and in a subsequent third step is deprotected to yield the free side chains and α-amino function, preferably it is partly or entirely deprotected by acidolysis.

In a further embodiment of the present invention, a method of amidating e.g. a biotechnologically produced, unprotected peptide having a free α-carboxyl group is devised, comprising the steps of
a) Recovering and isolating said peptide from a concatemeric fusion protein of said peptide, which fusion protein may further comprise interspersed linker sequences,
b) derivatizing at least the α-amino function of the unprotected peptide with non-base-labile protection groups, preferably also protecting nucleophilic groups of individual amino acid side chains by non-base-labile protection groups,
c) preferably recovering the protected peptide by precipitation with water,
d) amidating the C-terminal carboxyl group by the method described in the preceding sections,
e) preferably isolating the amidated peptide,
f) and deprotecting the side chains and α-amino function.

All the above described, preferred embodiments drawn to peptides of the present invention likewise specifically and preferably apply to (C-terminally) amidating antimicrobial peptides, preferably to antimicrobial peptides, more and most preferably to ILRWPWWPWRRK and/or other indolicidin derivatives.

### EXAMPLES

Boc-protection of a 12mer (N to C terminus, OH indicating free α-carboxyl group:

ILRWPWWPWRRK-OH

Protection of the peptide was carried out essentially as described in published application US2003/0219854 A1, except for the fact that the product was recovered by precipitation instead of lyophylization. The Di-Boc-ILRWPWWPWRRK-OH (ESR-MS: m/z 1980) was used in the subsequent amidation without further purification. Bocylation was carried out using Di-tert.butyl dicarbonate (Boc₂O) in acetonitrile/1N NaOH/H₂O. The reaction mixture was stirred at room temperature, the pH was then adjusted until pH 4.6 and the organic solvents were removed under vacuum. The pH was then again adjusted to 2. Water was added and the suspension cooled down to 4° C. before isolation of the precipitated thus formed. The solide was washed in water and then dried in vacuo.

C-terminal amidation of Boc-ILRWPWWPWRRK(Boc)-OH (diBoc-peptide-OH)

HCTU (209 mg, 0.5 mmol) is added to a solution of diBoc-MBI1B7 (900 mg, 0.455 mmol), N-methylmorpholine (150 μl, 1.365 mmol) and NH₃/HOOBt (250 mg, 1.365 mmol) in DMF (36 ml) and stirred at 25° C. under nitrogen atmosphere. If required, the pH is adjusted with N-methylmorpboline to pH 8.5 (±0.5). The reaction mixture is stirred at 25° C. for 2 h and controlled to maintain a pH of 8.5 (±0.2).

DMF is then evaporated under reduced pressure (water bath temperature <50° C.). Water is added (90 ml) and the suspension kept at 4° C. for at least 2 h before isolation. The solid is filtered and the cale washed with water (twice 25 ml). The crude peptide is dried under vacuum for 15 h (temperature <50° C.). A white powder is obtained (910 mg) that is subjected for further analysis.

Analytical method: Derivatisation of single amino acids after complete hydrolysis of peptide

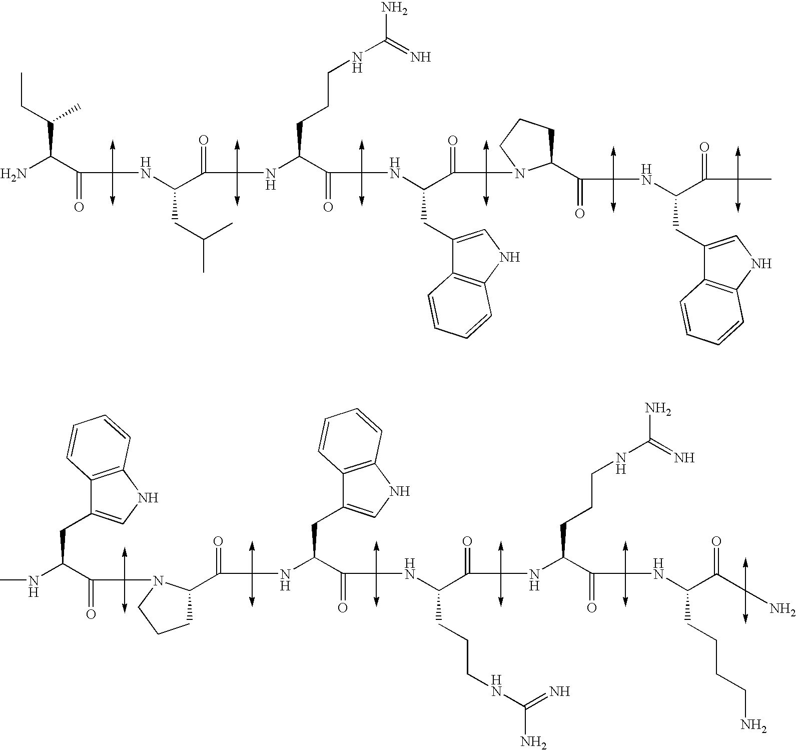

Using a 6N HCl treatment the peptide is hydrolysed into the single amino acids. During this treatment the C-terminal Lysine amide is converted into the corresponding carboxylic acid (Lys-OH) as described by Marfey et al. (Marfey, P., Carlsberg Res. Commun., 49, (1984), 591). Except possibly for Cys, such complete hydrolysis does not promote racemisation of amino acids. In short, the method devises to derivatize optical isomers of amino acids in the hydrolysate with FDAA (1-fluoro-2,4-dinitrophenyl-5-L-alanine). The simple derivatization procedure is completed within 90 minutes.

In the present context, the lysine derivatives can be easily separated and quantified by reverse phase HPLC. Derivatives have an absorption coefficient of ˜3×10⁴ and can be detected by UV at 340 nm with picomole sensitivity. First the L-Lys-NH₂ and the D-Lys-NH₂ were hydrolysed and derivatized.—Conversion rate and hence total yield (Conv. %) was determined by HPLC separation of the amidated diastereomers from the educt. The product retaining L-configuration at the terminal lysine-amid was identified by the method of Marfey et al., and the relative excess of the non-epimerised L-Lys product vs. the D-Lys product (% P) was determined by HPLC. An HPLC method was developed for the diastereoisomer analysis essentially as described in Marfey et al. and a good separation was obtained.

For the amidation of the same peptide, different combinations of coupling reagents and additives have been tested (Table I). For comparative purposes, where indicated exceptionally aqueous ammonia along with separate coupling additive was used instead of the ammonium salt of the coupling additive devised according to the present invention (Table I). ‘SM’ indicates the amount of Boc-ILRWPWWPWRRK(Boc)-OH reacted.

TABLE I

| SM [mg] | C. Rgt | Co-activ. | Base | Solvent | “NH₃” | Conv. % | % P |
|---|---|---|---|---|---|---|---|
| 26.0 | T3P | — | NMM | DMF | NH₃/HOOBt | 80 | 92.0 |
| 51.0 | HCTU | — | NMM | DMF | NH₃/HOOBt | 100 | 92.5 |
| 51.2 | HBTU | — | NMM | DMF | NH₃/HOOBt | 93 | 93.7 |
| 51.0 | EDC | Cl—HOBt | TMP | DMF | NH₃/HOOBt/CuCl₂ | 79 | 90.3 |
| 51.0 | EDC | Cl—HOBt | TMP | DMF | NH₃/HOOBt | 98 | 95.2 |
| 50.0 | IBCF | — | NMM | DMF | NH₃/HOOBt | 82 | 80.1 |
| 50.4 | IBCF | — | TMP | DMF | NH₃/HOSu | 88 | 82.2 |
| 50.9 | TCTU | — | NMM | DMF | NH₃/HOOBt | 89 | 94.2 |
| 50.1 | IBCF | — | NMM | DMF | Aqueous NH₃ | 85 | 52.6 |
| 304.0 | HCTU | — | NMM | DMF | NH₃/HOOBt | 94 | 94.3 |
| 49.8 | HCTU | — | NMM | DMF | NH₃/HOOBt | 100 | 94.8 |

The use of an ammonium salt of an co-activating reagent enhances the retention of the C-terminal amino acid configuration.

Deprotection of Boc-ILRWPWWPWRRK(Boc)-OH

Deprotection of 100 mg DiBoc-peptide takes place in 0.8 ml DMF to which 0.2 mL trifluoroacetic acid were added. The mixture is stirred for 30 min. at room temperature. The unprotected peptide-amide is then further purified by reverse phase HPLC and is finally lyophilized (ESI-MS: m/z 1779).

1. Method for amidating the free α-carboxyl group of an amino acid or preferably a peptide, comprising the first step of reacting said amino acid or peptide, with a peptide coupling reagent in an organic solvent in the presence of a first base and in the presence of an ammonium salt of at least one peptide coupling additive wherein the ammonium cation is selected from the group consisting of ammonia, primary amine and secondary amine and wherein the side chain and α-amino function of said amino acid or peptide are protected with non-base-labile protection groups.

2. Method for amidating the free α-carboxyl group of an amino acid or preferably a peptide, comprising the step of reacting said amino acid or peptide with a peptide coupling reagent in the presence of a first base and in the presence of an ammonium salt of at least one peptide coupling additive wherein the ammonium cation is selected from the group consisting of ammonia, primary amine and secondary amine and wherein the side chain and α-amino function of said amino acid or peptide are protected with non-base-labile protection groups with the exception of the ω-carboxyl groups of individual aspartyl- or glutamyl-residues and wherein said ω-carboxyl groups are amidated concomitantly with the free α-carboxyl group in the reaction.

3. Method according to claim 1 or 2, characterized in that the first base is a weak base whose conjugated acid has a pKa value of from pKa 7.5 to 15, preferably of from pKa 7.5 to 10.

4. Method according to one of the preceding claims, characterized in that the coupling reagent is a coupling reagent other than a carbodiimide.

5. Method according to claim 4, characterized in that the coupling reagent is selected from the group consisting of uronium salts and phosphonium salts of the benzotriazol capable of activating said α-carboxyl group and that the reaction is carried out in the presence of a weak first base.

6. Method according to claim 5, characterized in that the uronium or phosphonium salt coupling reagent is selected from the group consisting of HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazol-1-yl-oxy-tripyrrolidinophosphoniumhexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethylurollium tetrafluoroborate), HAPyU (O-(benzotriazol-1-yl)oxybis-(pyrrolidino)-uronium hexafluorophosphate.

7. Method according to claim 5, characterized in that the weak base is 2,4,6-trialkylpyridine or N-alkyl-morpholine, preferably with the alkyl being straight or branched C1-C4 alkyl.

8. Method according to claim 7, characterized in that the weak base is N-methylmorpholine or is 2,4,6-trimethylpyridine.

9. Method according to one of the preceding claims, characterized in that the organic solvent is an aprotic organic solvent, preferably is a polar aprotic organic solvent.

10. Method according to claim 9, characterized in that the solvent is selected from the group consisting of acetonitrile, dimethylsulfoxid, dimethylacetamid, dichloromethane and N,N-dimethyl-formamide, preferably it is N,N-dimethylformamide.

11. Method according to one of the preceding claims, characterized in that the coupling reagent additive is a nucleophilic N-hydroxy compound capable of forming activated esters.

12. Method according to claim 11, characterized in that the coupling reagent additive is selected from the group consisting of N-hydroxy-succinimide, N-hydroxy-3,4-dihydro-4-oxo-1,2,3-benzotriazine, 1-hydroxy-7-azabenzotriazole and N-hydroxy-benzotriazole.

13. Method according to claim 12, characterized in that the coupling reagent additive is hydroxy-3,4-dihydro-4-oxo-1,2,3-benzotriazine.

14. Method according to one of the preceding claims, characterized in that the pH is controlled during reaction to be in the range of about pH 8 to 9, preferably is controlled at about pH 8.5.

15. Method according to claim 4, characterized in that the method is carried out with a carbodiimide as the coupling reagent and, preferably, in the presence of a protonated N-hydroxy benzotriazol as a second coupling additive.

16. Method according to claim 15, characterized in that the carbo-diimide coupling reagent is selected from the group consisting of diisopropyl-carbodiimide, dicyclohexyl-carbodiimide and 1-ethyl-3-(3-dimethylaminopropyl)-carbodiimide), preferably is 1-ethyl-3-(3-dimethylaminopropyl)-carbodiimide.

17. Method according to claim 14, characterized in that the coupling uronium or phosphonium salt reagent is an uronium salt reagent and preferably is HCTU, TCTU or HBTU and more preferably is used in the reaction in combination with an ammonium salt of N-hydroxy-3,4-dihydro-4-oxo-1,2,3-benzotriazine.

18. Method according to one of the preceding claims, characterized in that the ammonium cation is ⁺H₂NR1R2 with R1,R2 each independently being H or being C1-C10, preferably being C1-C5, aliphatic or alicyclic hydrocarbon.

19. Method according to claim 18, characterized in that R1 is H and R2 is methyl, ethyl, propyl or isopropyl or in that R1,R2 are both H.

20. Method according to one of the preceding claims, characterized in that in a second step after the first reaction step, the amidated, protected amino acid or preferably peptide is isolated and in a third step is deprotected to yield the free side chains and α-amino function, preferably is partly or entirely deprotected by acidolysis.

21. Method for amidating the free α-carboxyl group of an ILRWPWWPWRRK, comprising the first step of reacting said ILRWPWWPWRRK with a peptide coupling reagent in an organic solvent in the presence of a first base and further in the presence of an ammonium salt of at least one peptide coupling additive wherein the ammonium cation is selected from the group consisting of ammonia, primary amine and secondary amine and wherein the side chain and α-amino function of said ILRWPWWPWRRK are protected with non-base-labile protection groups.

22. Method for amidating the free α-carboxyl group of an antimicrobial peptide, preferably a cationic antimicrobial peptide, comprising the first step of reacting said peptide with a peptide coupling reagent in an organic solvent in the presence of a first base and further in the presence of an ammonium salt of at least one peptide coupling additive wherein the ammonium cation is selected from the group consisting of ammonia, primary amine and secondary amine and wherein the side chain and α-amino function of said peptide are protected with non-base-labile protection groups.

23. Method according to claim 21 or 22, characterized in that the base is a weak base whose conjugated acid has a pKa value of from pKa 7.5 to 10.

24. Method according to one of the preceding claims, characterized in that the coupling reagent is a coupling reagent other than a carbodiimide.

25. Method according to claim 21 or 22, characterized in that the coupling reagent is selected from the group consisting of uronium salts and phosphoniun salts of a benzotriazol capable of activating said α-carboxyl group and that the reaction is carried out in the presence of a weak first base.

26. Method according to claim 25, characterized in that the uronium or phosphonium salt coupling reagent is selected from the group consisting of HBTU (O-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazol-1-yl-oxy-tripyrrolidinophosphonium hexafluorophosphate), PyAOP, HCTU (O-(1H-6-chloro-benzotriazole-1-yl)-1,1,3,3-tetramethyluronium hexafluorophosphate), TCTU (O-1H-6-chlorobenzotriazole-1-yl)-1,1,3,3-tetramethyluronium tetrafluoroborate), HATU (O-(7-azabenzotriazol-1-yl)-1,1,3,3-tetramethyluronium hexafluorophosphate), TATU (O-(7-azabenzotriazol-1-yl)-1,1,3,3-tetramethyluronium tetrafluoroborate), TOTU (O-[cyano(ethoxycarbonyl)methyleneamino]-N,N,N′,N″-tetramethyluronium tetrafluoroborate), HAPyU (O-(benzotriazol-1-yl)oxybis-(pyrrolidino)-uronium hexafluorophosphate.

27. Method according to claim 25, characterized in that the weak base is 2,4,6-trialkylpyridine or N-alkyl-morpholine, preferably with the alkyl being straight or branched C1-C4 alkyl.

28. Method according to claim 27, characterized in that the weak base is N-methylmorpholine or is 2,4,6-trimethylpyridine.

29. Method according to claim 28, characterized in that the weak base is Hückel-base, N,N-diisopropylethylamine, 2,4,6-trimethylpyridine or N-alkyl-morpholine, preferably is N-alkylmorpholine with the alkyl being straight or branched C1-C4 alkyl, most preferably is N-methylmorpholine.

30. Method according to one of the preceding claims, characterized in that the organic solvent is an aprotic organic solvent, preferably is a polar aprotic organic solvent.

31. Method according to claim 30, characterized in that the solvent is selected from the group consisting of acetonitrile, dimethylsulfoxid, diemethylacetamide, dichloromethane and N,N-dimethyl-formamide, preferably it is N,N-dimethylformamide.

32. Method according to one of the preceding claims, characterized in that the coupling reagent additive is a nucleophilic N-hydroxy compound capable of forming activated esters.

33. Method according to claim 32, characterized in that the coupling reagent additive is selected from the group consisting of N-hydroxy-succinimide, N-hydroxy-3,4-dihydro-4-oxo-1,2,3-benzotriazine, 1-hydroxy-7-azabenzotriazole and N-hydroxy-benzotriazole.

34. Method according to claim 33, characterized in that the coupling reagent additive is hydroxy-3,4-dihydro-4-oxo-1,2,3-benzotriazine.

35. Method according to one of the preceding claims, characterized in that the pH is controlled during reaction to be in the range of about pH 8 to 9, preferably is controlled at about pH 8.5.

36. Method according to claim 24, characterized in that the method is carried out with a carbodiimide as the coupling reagent and, preferably, in the presence of a protonated N-hydroxy benzotriazol.

37. Method according to claim 36, characterized in that the carbodiimide coupling reagent is selected from the group consisting of diisopropyl-carbodiimide, dicyclohexyl-carbodiimide and 1-ethyl-3-(3-dimethylaminopropyl)-carbodiimide), preferably is 1-ethyl-3-(3-dimethylaminopropyl)-carbodiimide.

38. Method according to claim 14, characterized in that the coupling uronium or phosphonium salt reagent is an uronium salt reagent and preferably is HCTU, TCTU or HBTU and more preferably is used in the reaction in combination with an ammonium salt of N-hydroxy-3,4-dihydro-4-oxo-1,2,3-benzotriazine.

39. Method according to one of the preceding claims, characterized in that the ammonium cation is ⁺H₂NR1R2 with R1,R2 each independently being H or being C1-C10, preferably being C1-C5, aliphatic or alicyclic hydrocarbon.

40. Method according to claim 39, characterized in that R1 is H and R2 is methyl, ethyl, propyl or isopropyl or in that R1, R2 are both H.

41. Method according to one of the preceding claims, characterized in that in a second step after the first reaction step, the amidated, protected ILRWPWWPWRRK or anti-microbial peptide, preferably anti-microbial cationic peptide is isolated and in a third step is deprotected to yield the free side chains and α-amino function, preferably is partly or entirely deprotected by acidolysis.


SEQUENCE LISTING (1 sequence)

SEQ ID NO: 1 - protein, 12 aa, Artificial
  Indolicidin derivative
ILRWPWWPWRRK